(19) United States
(12) Patent Application Publication
(10) Pub. No.: US 2011/0105654 A1
(43) Pub. Date: May 5, 2011

(54) Method for Producing a Compound with at Least One at Least Monosubstituted Amino Group

(75) Inventors: Rene Dicke, Leonding (AT); Martin Burger, Traunstein (DE)

(73) Assignee: BOREALIS AGROLINZ MELAMINE GMBH, Linz (AT)

(21) Appl. No.: 12/934,595

(22) PCT Filed: Mar. 27, 2009
(86) PCT No.: PCT/EP09/02439

§ 371 Date: Dec. 20, 2010

(30) Foreign Application Priority Data
Mar. 31, 2008 (DE) 102008016967.6

(51) Int. Cl. C08K 5/3492; C07D 251/70; C07C 273/18; C08K 5/21

(52) U.S. Cl. 524/100; 544/196; 564/60; 524/213

(57) ABSTRACT

The invention relates to a method for producing a compound having at least one mono substituted amino group. According to the invention, a starting substance supporting at least one amino group as reacted with a reagent supporting a first functional group and at least one other functional group in a reaction mixture. The first functional group is a hydroxyl group and the at least one other functional group is selected from hydroxyl groups, mercapto groups, carboxylic acid groups, carboxylic acid ester groups, carboxylic acid amide groups, keto groups, carbonate groups, halogens, epoxy groups and carbamate groups. If several other functional groups are present, the groups are selected independently of one another from the aforementioned class. Melamine, urea and guanamine are, in particular, reacted with glycol, glycerine and hydroxyl acetic acid derivatives.

The invention relates to a method for producing a compound with at least one at least one substituted amino group according to the preamble of claim 1 and the use of such a compound according to claim 24 or 26.

JP 2000-063360 A describes a method for alkylation of melamine in which melamine is reacted with an alcohol at high temperatures in the presence of a metal catalyst on a microporous carrier. Thereby, preferably lower alkylated products are obtained. In this method a low selectivity of the alkylated products on the one hand and the formation of cyanic acid esters as side products by hydrolysis or substitution of the amino groups on the other hand are recognized. The reaction occurs under a nitrogen, argon, hydrogen or carbon monoxide atmosphere.

EP 0 711 760 A1 describes an alkylation of melamine by reacting melamine in the presence of a catalyst and an atmosphere of argon, nitrogen, carbon monoxide or a mixture of hydrogen and carbon monoxide. No complete conversion of the educts and no selectivity in respect to single products are achieved.

EP 1 057 821 A1 describes the alkylation of melamine with alcohols in the presence of catalysts and a nitrogen or hydrogen atmosphere. No complete conversion of the educts and no selectivity in respect to single products are achieved.

Shinoda et al. (Appl. Catalysis A: General 194-195 (2000), 375-381) describes a methylation of melamine starting from methanol. A metal with an acidic carrier is used for the catalysis and the reaction is carried out under protection gas (argon) or hydrogen atmosphere. The reaction achieves a complete conversion, however only after very long reaction times. The formed product spectrum contains different substituted methyl melamines.

The object of the resent invention is to provide compounds with at least one monosubstituted amino group, whereby a further reaction of the formed compounds should also be possible at the substituents.

This object is being solved with a method with the features of claim 1. Accordingly, a starting substance having at least one amino group is reacted with a reagent substituted by a hydroxyl group or by at least on further functional group. The further functional group is selected from the class comprising hydroxyl groups, mercapto groups, carboxylic acid groups, carboxylic acid ester groups, carboxylic acid amide groups, keto groups, carbonate groups, halogens, epoxy groups and carbamate groups. In case more than one further functional group is present in the reagent, the single further functional groups are independently from each other selected from said class. The starting substance and the reagent are (optionally exclusive) part of a reaction mixture.

In a variant the class from which the functional group is selected comprises only hydroxyl groups. In a further variant the class from which the functional group is selected comprises only mercapto groups. In a further variant the class from which the functional group is selected comprises only carboxylic acid groups. In a further variant the class from which the functional group is selected comprises only carboxylic acid ester groups. In a further variant the class from which the functional group is selected comprises only carboxylic acid amide groups. In a further variant the class from which the functional group is selected comprises only keto groups. In a further variant the class from which the functional group is selected comprises only carbonate groups. In a further variant the class from which the functional is selected comprises only halogens. In a further variant the class from which the functional group is selected comprises only epoxy groups. In a further variant the class from which the functional group is selected comprises only carbamate groups. In a further variant the class from which the functional group is selected comprises any combination of the previously mentioned groups.

The amino group of the starting substance has at least one hydrogen atom directly linked to the nitrogen. This means that this amino group can be unsubstituted or monosubstituted. After the reaction with the reagent R-OH the moiety R is bound to the nitrogen atom of the amino group instead of the original hydrogen atom. In other words, the method according to the invention is a method for derivatizing an amino group.

Surprisingly, it was found that a catalytic or non-catalytic derivatization, in particular alkylation with substituted alkyl moieties, of the amino group is possible with a reagent having at least one hydroxyl group, even if the reagent carries further functional groups. By inserting multiple substituents in the respective starting substance a high selectivity in respect to the distribution of the substituents on the single amino groups of the starting substance is also achieved.

In a variant the reaction occurs in the presence of a further substance which is selected from the class comprising ammonia, nitrogen, argon, hydrogen and carbon monoxide, so that in particular the gas phase of the reaction mixture contains this further substance in gaseous form. Also, the further substance can, for instance in dissolved form, be part of the reaction mixture. The reaction mixture can be for instance a liquid, dispersion or a suspension.

The molar ratio between the further substance and reagent is in a variant 0.1 to 2, in particular 0.5 to 1.5 and especially in particular 0.8 to 1.3.

Ammonia is in particular suitable as a further substance since it has been shown that the use of ammonia as gas or as solution provides an almost complete suppression of side products and leads simultaneously to a significant increase of the selectivity of the derivatization reaction. The side product formation by hydrolysis can be in particular significantly reduced or suppressed.

The conversion occurs in an embodiment at a total pressure of ca. 1 to ca. 200 bar, in particular at ca. 40 bar to ca. 180 bar and especially in particular at ca. 60 bat to ca. 140 bar. The total pressure consists thereby of the partial pressure of the gases contained in the gas phase. The further substance can thereby present at least a part of the gases present in the gas phase. Also the reagent can be at least partially a gas which is contained in the gas phase.

In a variant the conversion occurs under the influence of a catalyst for increasing the conversion rates in order to be able to keep the reaction temperatures on a low level, for increasing the selectivity in respect to the products and/or to reduce the reaction times.

The catalyst comprises in an alternative embodiment a metal or a metal oxide. Also mixtures of different metals and/or metal oxides are possible.

The catalyst is in particular a metal from the 8^{th}, 9^{th} or 10^{th} IUPAC-group (VIII. subgroup) of the periodic system. Amongst others iron, cobalt, nickel, ruthenium, rhodium, palladium and platinum belong to said group.

The used amount of a catalyst is in a variant in the area of 0.001 to 20 Mol-%, in particular 0.01 to 10 Mol-%, in particular 0.1 to 1 Mol-% and especially particular 0.1 to 0.5 Mol-% in each case in respect to the amount of the substance of the compound to be converted.

In a further embodiment the catalyst has a carrier material. If for instance a porous carrier material is used the surface of the catalyst can be increased and the amount of the catalytic active metal or metal oxide can be reduced. Suitable carrier materials are for instance zeolithes, alumosilicates, alumophosphates, metal oxides, silicates, layered silicates, aluminium oxide, silicium dioxide and carbon.

Examples for zeolithes are Beta-zeolithe (BEA), Y-zeolithe, faujasite, mordenite, ZSM-5, zeolithe X, zeolithe A.

Examples for layered silicates are montmorillonit, mordenite, bentonite, kaolinite, muskovite, hectorite, fluorhectorite, kanemite, revdite, grumantite, ilerite, saponite, beidelite, nontronite, stevensite, laponite, taneolite, vermiculite, halloysite, volkonskoite, magadite, rectorite, kenyaite, sauconite, borfluorphlogopite and/or synthetic smectites.

The catalysts can be used as powder, molded and monolith catalysts, latter ones for instance on honeycomb structures. For the conversion for instance fixed bad reactors, fluidized bed reactors, steered tank reactors or tubular reactors are used. The reactors are operated in a variant for the conversion at temperatures of ca. 100° C. to ca. 300° C., in particular ca. 150° C. to ca. 250° C. and especially in particular ca. 180° C. to ca. 240° C.

The required reaction times are in an embodiment below 100 hours, in particular between ca. 1 Minute and ca. 50 hours, in particular between ca. 1 hour and ca. 20 hours, and especially in particular between ca. 4 hours and ca. 12 hours.

In an alternative embodiment the catalyst is being separated after complete conversion that means after the reaction has ended or is being interrupted from the reaction mixture for instance by filtration and is further worked up.

In a variant the starting substance is a triazine derivative of the general formula (I) or a urea or urea derivative of the general formula (II).

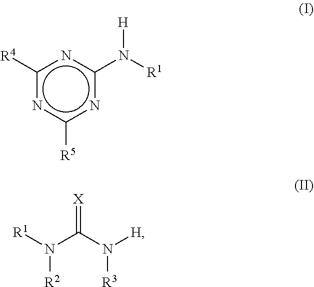

whereby
   R⁴ and R⁵ mean independently from each other Q¹ or a moiety of the formula R⁶—N—R⁷ or R⁸—N—R⁹ connected with its central nitrogen atom to the triazine ring of the structure of formula (I), whereat
      Q¹ means a linear or branched C₁-C₃₀-alkyl or a cyclic substituent in form of a C₅-C₂₀-cycloalkyl, a C₅-C₂₀-aryl, a C₁-C₂₀-aikylsubstituted C₅-C₂₀-aryl or an amide of a cyclic unsaturated carboxylic acid, whereat the C₁-C₃₀-alkyl or the cyclic substituent can be interrupted by one or multiple oxygen atoms, sulphur atoms, substituted nitrogen atoms and/or by one or multiple groups of the type —C(O)O—, —OC(O)—, —C(O)— and/or —OC(O)O—,
   R¹, R², R³, R⁶, R⁷, R⁸ and R⁹ mean independent from each other H, linear or branched C₁-C₂₀-alkyl, C₅-C₂₀-cyclo alkyl, C₅-C₂₀-aryl, C₁-C₂₀-alkyl substituted C₅-C₂₀-aryl, which in each case can be interrupted by one or multiple oxygen atoms, sulphur atoms and/or substituted nitrogen atoms and/or by one or multiple groups of the type —C(O)O—, —OC(O)—, —C(O)— and/or —OC(O)O— and/or can be functionalized by one or multiple hydroxyl groups and/or mercapto groups, and
   X means O or S.

In a further embodiment a compound of the general formula R¹⁰—OH is being used as reagent so that at least one hydroxyl group is present as first functional group in the reagent, whereby
R¹⁰ means a linear or branched C₁-C₂₀-alkyl, C₅-C₂₀-cycloalkyl or C₁-C₂₀-alkyl substituted C₅-C₂₀-aryl, which in each case can be interrupted by one or multiple oxygen atoms, sulphur atoms, substituted nitrogen atoms and/or by one or multiple groups of the type —C(O)O—, —OC(O)—, —C(O)— and/or —OC(O)O— and has at least one further functional group which is selected from the class comprising hydroxyl groups, mercapto groups, carboxylic acid groups, carboxylic acid ester groups, carboxylic acid amide groups, keto groups, carbonate groups, halogen, epoxy groups and carbamate groups.

In an embodiment at least one hydroxyl group of the reagent is bound to the alkyl moiety and not to the aryl moiety, if R¹⁰ means a C₁-C₂₀-alkylsubstituted C₅-C₂₀-aryl which can be interrupted or substituted as above. Thereby it can also be provided that all hydroxyl groups are bound to the alkyl moiety and not to the aryl moiety. In this manner the formation of arylated triazine or urea derivatives can be avoided in favour of the formation of arylsubstituted alkylated derivatives.

Examples for possible triazine derivatives as starting substances are melamine, benzoguanamine, acetoguanamine, 2,4,6-Tris-(2-hydroxyethyl)amino-1,3,5-triazine, 2-succinimido-4,6-diamino-1,3,5-triazine, 2,2-dimethylamino-4,6-diamino-1,3,5-triazine, 2,2-dibutylamino-4,6-diamino-1,3,5-triazine and 2,4,6-Tris-methylamino-1,3,5-triazine. Examples for urea derivatives as starting substances are hydroxyethyl urea and ethylene urea. Also non-derivatized urea can be used as starting substance.

Polyalcohols (under which also dials and oligools are to be understood), thiols and other hydroxyl compounds with at least one further functional group can be for instance used as reagents.

Examples for suitable polyalcohohols are ethylenglycol, diethylenglycol, glycerol, trimethylolpropane, pentaerythrit, tripropylenglycol, trisopropanolamine, triethanolamine, hexandiol, butandiol and glycerolmonostearat.

Examples for suitable thiols are mercaptoethanol, mercaptopropanol, mercaptomethylbutanol and mercaptohexanol.

Examples for hydroxyl compounds with at least one further functional group are hydroxyl acetic acid, hydroxyl acetic acid methylester, hydroxyl acetic acid ethylester, 2,2-Bis(hydroxymethyl)propionic acid, methyl-2,2dimethyl-3-hydroxypropionate, tert-butyl-3-hydroxypropionate and N,N-diethyl-2-hydroxyacetamide.

In an embodiment of the method it is sufficient in case of short-chain alcohols as educts for the derivatization of the starting substance to apply the alcohol in excess in order to use said alcohol also as a solvent. In a further variant inert solvents are used as solubilizer when using long-chain alcohols (more than 8, 10 or 12 C-atoms) in order to achieve a better conversion. Basically, solubilizers can also be used with short-chain alcohols if those are for instance highly branched and have therefore a higher viscosity. The application of a solubilizer is always appropriate in such cases when the mixture cannot be stirred anymore without a solubilizer.

Examples for such solubilizers are tetrahydrofurane, diethylether, dimethoxymethane, dimethoxyethane, diethoymethane, diethoxethane, ethylenglycoldiethylether, ethylenglycoldibutylether, diethylenglycoldiethylether, dioxan, benzene, toluene, xylene, mesitylene, cumen, chlorbenzene, pentane, hexane, cyclohexane, heptane, octane, acetonitrile, methylacetate, ethylacetate, menthylbenzoate, N,N-dimethylformamide, N,N-dimethylacetamide, N-methylpyrrolidone, 1,3-dimethylimidazolidinone.

It is recommended in case of a reagent with multiple hydroxyl groups to work strongly diluted in order to avoid side reactions by which oligomeric or polymeric compounds are formed from multiple triazine or urea units. For instance molecules can be formed from two triazine units in which the two triazine units are connected with each other via an ethylene bridge if a high concentration is used and glycol (1,2-ethandiol) is used as reagent. This is not desirable and can be avoided by reacting at a lower dilution.

In a variant the formed compound is a triazine derivative of the general formula (III) or urea derivative of the general formula (IV):

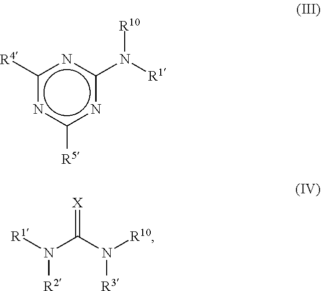

whereby
   R^{4′} and R^{5′} mean independently from each other Q¹ or a moiety of the formula R^{6′}—N—R^{7′}— or R^{8′}—N—R^{9′} being connected with its central nitrogen atom to the triazine ring of the structure of the formula (Ill), whereat
      Q¹ means a linear or branched C₁-C₃₀-alkyl or a cyclic substituent in form of a C₅-C₂₀-cyclo alkyl, a C₅-C₂₀-aryl, a C₁-C₂₀-alkyl substituted C₅-C₂₀-aryl or an amide of a cyclic unsaturated carboxylic acid, whereat the C₁-C₃₀-alkyl or the cyclic substituent can be interrupted by one or multiple oxygen atoms, sulphur atoms, substituted nitrogen atoms and/or by one or multiple groups of the type —C(O)O—, —OC(O)—, —C(O)— and/or —OC(O)O—,
   R^{1′}, R^{2′}, R^{3′}, R^{6′}, R^{7′}, R^{8′} and R^{9′} mean independently from each other,
      R¹⁰,
      a covalent bond bound to the moiety R¹⁰ being bound to the same nitrogen atom of the triazine derivative or urea derivative so that a cyclic structure is formed from the nitrogen atom and the moiety R¹⁰,
      H or
      linear or branched C₁-C₂₀-alkyl, C₅-C₂₀-cyclo alkyl, C₅-C₂₀-aryl, C₁-C₂₀-alkyl substituted C₅-C₂₀-aryl, which in each case can be interrupted by one or multiple oxygen atoms, sulphur atoms and/or substituted nitrogen atoms and/or by one or multiple groups of the type —C(O)O—, —OC(O)—, —C(O)— and/or —OC(O)O— and/or can be functionalized by one or multiple hydroxy groups and/or mercapto groups,
   R¹⁰ means a linear or branched C₁-C₂₀-alkyl, C₅-C₂₀-cyclo alkyl or C₁-C₂₀-alkyl substituted C₅-C₂₀-aryl, which in each case can be interrupted by one or multiple oxygen atoms, sulphur atoms, substituted nitrogen atoms and/or by one or multiple groups of the type —C(O)O—, —OC(O)—, —C(O)— and/or —OC(O)O—, and
      a) has at least one further functional group, which is selected from the class comprising hydroxyl groups, mercapto groups, carboxylic acid groups, carboxylic acid ester groups, carboxylic acid amide groups, keto groups, carbonate groups, halogens, epoxy groups carbamate groups. or
      b) thereby forms a cyclic structure with two covalent bonds bonded to the same nitrogen atom of the compounds of the general formula (Ill) or (IV), whereat one or the two covalent bonds is provided by one of the moieties R^{1′}, R^{2′}, R^{3′}, R^{6′}, R^{7′}, R^{8′} or R^{9′} and whereat in the moiety R¹⁰ at least one further functional group can be present, which is selected from the class comprising hydroxyl groups, mercapto groups, carboxylic acid groups, carboxylic acid ester groups, carboxylic acid amide groups, keto groups, carbonate groups, halogens, epoxy groups and carbamate groups, and
   X means O or S.

The term “further functional group” in respect to the meaning of the moiety R¹⁰ in a compound of the general formula (III) or (IV) is thereby to be understood such that in the moiety R¹⁰ at least one functional group of the mentioned class is present or can be present, whereby not necessarily a further functional group is present in this moiety. The hydroxyl group originally present in moiety R¹⁰ is no longer present after the completed reaction with the triazine derivative of the general formula (I) or the urea derivative of the general formula (II) to a compound of the general formula (III) or (IV).

A cyclic structure according to variant b) is formed for instance if a reagent is used that carries at least two hydroxyl groups which react in each case with the same nitrogen atom of the starting compound.

In an embodiment of the method at least one moiety, in particular at least two moieties, in particular at least three moieties, in particular at least four moieties, in particular at least five moieties of the moieties R^{1′}, R^{2′}, R^{3′}, R^{6′}, R^{7′}, R^{8′} and R^{9′} have only the meaning of the moiety R¹⁰ and in particular not the meaning of H. This is in particular the case if the corresponding moieties R^{1′}, R^{2′}, R^{3′}, R^{6′}, R^{7′}, R^{8′} and R^{9′} have the meaning of a hydrogen moiety in the starting substance. In other words, several different amino groups of the formed compound (for instance one, two or three amino groups) can be substituted with the moiety R¹⁰ to a different degree (for instance once or twice) independently from each other.

Using suitable experimental parameters essentially pure N,N′-dihydroxyalkyl compounds, N,N′,N″-trihydroxyalkyl compounds, N,N′,N″-trihydroxyalkyl-N,N′,N″-trialkyl compounds, N,N,N′,N″-tetrahydroxyalkyl compounds or N,N,N′,N′,N″,N″-hexahydroxyalkyl compounds can be synthesized.

Using suitable experimental parameters also essentially pure N,N′-dicarbonylalkyl compounds, N,N′,N″-tricarbonylalkyl compounds, N,N′,N″-tricarbonylalkyl-N,N′,N″-trialkyl compounds, N,N,N′,N″-tetracarbonylalkyl compounds or N,N,N′,N′,N″,N″-hexacarbonylalkyl compounds can be synthesized.

In an embodiment the formed compound is selected from the group comprising melamine-N-monoalkylcarboxylic acid, melamine-N,N′-dialkylcarboxylic acid, melamine-N,N′,N″-trialkylcarboxylic acid, melamine-N,N,N′,N″-tetra-alkylcarboxylic acid, melamine-N,N,N′,N′,N″-penta-alkylcarboxylic acid and melamine-N,N,N′,N,N″,N″-hexa-alkylcarboxylic acid.

In a further embodiment the formed compound is selected from the group comprising urea-N-mono(alkylcarboxylic acid), urea-N,N′-di(alkylcarboxylic acid), urea-N,N,N′-tri(alkylcarboxylic acid), urea-N,N,N′,N′-tetra(alkylcarboxylic acid).

In an alternative embodiment the formed compound is selected from the group comprising benzoguanamine-N-mono(alkylcarboxylic acid), benzoguanamine-N,N′-di(alkylcarboxylic acid), benzoguanamine-N,N,N′-tri(alkylcarboxylic acid), benzoguanamine-N,N,N′,N′-tetra(alkylcarboxylic acid).

In a further alternative embodiment the formed compound is selected from the group acetoguanamine-N-mono(alkylcarboxylic acid), acetoguanamine-N,N′-di(alkylcarboxylic acid), acetoguanamine-N,N,N′-tri(alkylcarboxylic acid), acetoguanamine-N,N,N′,N′-tetra(alkylcarboxylic acid).

In a further embodiment the formed compound is selected from the group comprising N-(Hydroxyalkyl)-melamine, N,N′-Di-(hydroxyalkyl)-melamine, N,N′,N″-Tris-(hydroxyalkyl)-melamine, N,N,N′,N″-Tetra-(hydroxyalkyl)-melamine, N,N,N′,N′,N″-Penta-(hydroxyalkyl)-melamine and N,N,N′,N′,N″,N″-Hexa-(hydroxyalkyl)-melamine.

In an embodiment the formed compound is selected from the group comprising melamine-N-mono(alkylcarboxylic acid-alkylester), melamine-N,N′-di(alkylcarboxylic acid-alkylester), melamine-N,N′,N″-tri(alkylcarboxylic acid-alkylester), melamine-N,N,N′,N″-tetra(alkylcarboxylic acid-alkylester), melamine-N,N,N′,N′,N″-penta(alkylcarboxylic acid-alkylester) and melamine-N,N,N′,N′,N″,N″-hexa(alkylcarboxylic acid-alkylester).

In particular methyl, ethyl, butyl and/or hexyl moieties (or their mixtures) can be used as alkyl moiety, respectively, but also all other meanings provided above for the moiety R¹⁰. All moieties R¹⁰ comprising hydroxyl groups, for instance 2-hydroxyethyl-, hydroxypropyl- and/or hydroxyethoxyethyl moieties can be used as hydroxyalkyl moiety.

By the means of the following reaction equations an exemplary embodiment of the claimed method shall be explained in more detail. Thereby “T” means an increased temperature compared to the room temperature and “p” means an increased pressure in respect to the standard air pressure (specific parameter embodiments or reaction conditions and meanings of the moieties Rⁿ are explained further above):

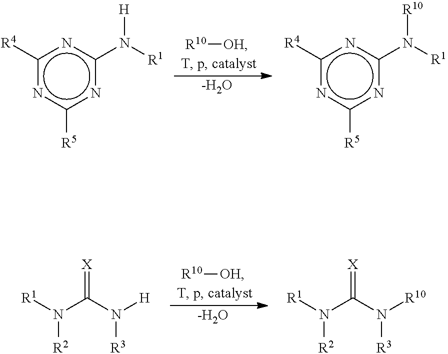

Due to the conversion of the starting substance a hydrogen atom bound to the nitrogen of an amino group is replaced by the moiety R¹⁰ of the used reagent; a derivatization of the amino group occurs. After completion of the reaction the derivatized amino group has at least one substituent being different from a hydrogen atom, namely R¹⁰. Depending on the reaction conditions also further moieties Rⁿ, which previously had the meaning of a hydrogen atom, can be replaced by the moiety R¹⁰. In this manner compounds with different degrees of substituted amino groups can be obtained.

Due to the obtained purity the derivatized compounds obtained according to this method as for instance derivatized amino triazines and derivatized ureas can be used as formaldehyde resins. Under the meaning “formaldehyde resin” a resin made of formaldehyde and the corresponding formed compound is to be understood. These formaldehyde resins have specific properties in respect to rheology, hydrophilicity or lipophilicity and surface properties. They are in particular suitable for application in the area of the laminate coating of the wood processing industry.

In particular, alkylated compounds as for instance the trihydroxyalkyl melamine (N,N′,N″-trihydroxyalkyl melamine) or other hydroxymelamines with at least three hydroxyl groups whereby an alkyl moiety can basically be substituted with more than one hydroxyl group, or melamine-N,N′,N″-trialkylcarboxylic acid or other alkylcarboxylic acid melamines with at least 3 carboxyl groups, whereby an alkyl moiety can generally be substituted with more than one carboxylic group are also suitable as cross linker. Further areas of application of the formed compounds, in particular, of the hydroxyalkylated compounds as for instance the hydroxyalkylated aminotriazine and the hydroxyalkylated urea, are the area of additives for plasticization, the area of flame retardant additives, the area of comonomers for a polyurethane and the area of agrochemicals.

If longer-chain lipophilic moieties as for instance alkyl moieties are bound to the starting substance carrying an amino group, the hydrophobicity of the formed compound is higher than the one of the starting substance. If the further functional group in the alkyl moiety to be inserted has additionally a polarity, the hydrophilicity of the formed compound (at least partially local) can be however increased compared to the starting compound. Then the compounds obtained by the claimed method are in particular suitable for use in a mixture with a polyolefin, in particular, a polyethylene (polyethen) of polypropylene (polypropene), since they allow for better interactions of the polyolefin with the surfaces to be contacted or other substances. In this manner for instance the flame retardant properties or surface properties (in respect to an improved adhesion compared to a coating to be applied) of an object consisting of the mixture of the polyolefin and the formed compound can be improved compared to an object consisting of an unmodified polyolefin.

It is also possible to insert an alcohol with at least three carbon atoms in one of the starting compounds and to create by subsequent water elimination a double bond in the substituent of the formed compound which can than be grafted onto a polyolefin.

Further details of the invention are explained by the means of the following examples. If nothing else is explicitly stated all percentages in the examples as well as in the other parts of the description and the claims have to be understood as weight percentages.

### EXAMPLE 1

In a 500 ml stirred autoclave 5.0 g melamine, 30 g hydroxyacetic acid, 100 ml dimethoxyglycol and 8 g of Ni/Y-zeolithe catalyst are intensively mixed so that the catalyst does not sink to the bottom. After closing the autoclave it is heated up to 180° . After 6 hours reaction time the reaction is aborted and the autoclave is cooled and depressurized. The solution is separated from the catalyst via a filter. The filtrate was taken up in 400 ml water/ethanol (1:1) and the pH is adjusted to 6. The precipitated product is being sucked off, washed and dried at 40° C. in a vacuum drying cabinet. In this manner 5.7 g product were isolated. A determination of the composition of the product by the means of quantitative HPLC provided 8% melamine-N-mono acetic acid, 12% melamine-N,N′-diacetic acid, 30% melaine-N,N′,N″-triacetic acid, 26% melamine-N,N,N′,N″-tetraacetic acid, 14% melamine-N, N, N′,N′,N″-pentaacetic acid and 10% melamine-N,N,N′,N′,N″,N″-hexaacetic acid.

### EXAMPLE 2

In a 500 ml stirred autoclave 5.0 g melamine, 107 g hydroxyacetic acid methyl ester and 10 g of Ru/BEA catalyst are intensively mixed so that the catalyst does not sink to the bottom. After closing the autoclave 50 g ammonia are pressed into the reactor and are heated to 230° C. After 4 hours reaction time the reaction is aborted and the autoclave is cooled and depressurized. The cooled solution is being separated from the catalyst via a filter and concentrated in high vacuum. In this manner 6.5 g product were isolated. The determination of the composition of the product by the means of quantitative HPLC provided 20% melamine-N-mono(acetic acid-methyl ester), 25% melamine-N,N′-di(acetic acid methyl ester), 30% melamine-N,N′,N″-tri(acetic acid methyl ester), 15% melamine-N,N,N′,N″-tetra(acetic acid methyl ester), 8% melamine-N,N,N′,N′,N″-penta(acetic acid methyl ester) and 2% melamine-N,N,N′,N′,N″,N″-hexa(acetic acid methyl ester).

### EXAMPLE 3

In a 500 ml stirred autoclave 5.0 g melamine, 300 g glycerol and 9 g of a Ru/BEA catalyst are intensively mixed so that the catalyst does not sink to the bottom. After closing the autoclave 50 g ammonia are pressed into the reactor and are being heated to 230° C. After 8 hours reaction time the reaction is aborted and the autoclave is cooled and depressurized. The cooled solution is being separated via a filter from the catalyst and is concentrated in a high vacuum. In this manner 6.9 g product were isolated. A determination of the composition of the product by the means of quantitative HPLC provided 12% N-(2,3-dihydroxypropyl)-melamine, 25% N,N′-Di(2,3-dihydroxyproyl)-melamine, 58% N,N′,N″-Tris(2,3-dihydroxypropyl)-melamine and 5% N,N,N′,N″-Tetra(2,3-dihydroxyalkyl)-melamine. The structural formula of N,N′-Di-(2,3-dihydroxypropyl)-melamine is shown in the following:

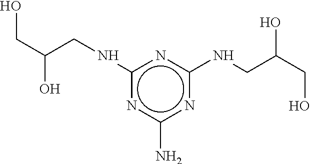

### EXAMPLE 4

In a 500 ml stirred autoclave 5.0 g melamine, 350 g N,N-diethyl-2-hydroxyacetamid and 10 g of a Ni/NiO catalyst are intensively mixed so that the catalyst does not sink to the bottom. After closing the autoclave it is being heated to 240° C. After 4 hours reaction time the reaction is aborted and the autoclave is cooled and depressurized. The cooled solution is being separated from the catalyst via a filter. The obtained product is dried in a vacuum drying cabinet at 40° C. A determination of the composition of the product via HPLC provided 22 area % melamine-N-mono-(N,N-diethyl-2-hydroxacetamid), 31 area % melamine-N,N′-di(N,N-diethyl-2-hydroxyacetamid), 45 area % melamine-N,N′,N″-tri(N,N-diethyl-2-hydroxyacetamid) and 2 area % melamine-N,N,N′,N″-tetra(N,N-diethyl-2-hydroxyacetamid). The area percentages reflect thereby the relative percentage of the peak area of a single HPLC peak on the total area of all detected HPLC peaks.

### EXAMPLE 5

In a 500 ml stirred autoclave 5.0 g urea, 150 g ethylenglycol and 5 g of a Ru/BEA catalyst are intensively mixed so that the catalyst does not sink to the bottom. After closing the autoclave it is heated to 200° C. After 4 hours reaction time the reaction is aborted and the autoclave is cooled and depressurized. The cooled solution is separated from the catalyst via a filter and is concentrated in a high vacuum. In this manner 3.5 g product were isolated. A determination of the composition of the product by the means of quantitative HPLC provided 76% N,N′-Di-(hydroxyethyl)-urea, 20% N-mono-(hydroxyethyl)-urea and 4% N,N,N′-Tri-(hydroxyethyl)-urea.

### EXAMPLE 6

In a 1000 ml stirred autoclave 5.0 g melamine, 400 g ethylenglycol and 9.5 g of a Ru/Mordenite catalyst are intensively mixed so that the catalyst does not sink to the bottom. After closing the autoclave it is heated to 220° C. After 6 hours reaction time the reaction is aborted and the autoclave is cooled and depressurized. The cooled solution is separated from the catalyst via a filter. The catalyst is being boiled with water at 100. The filtrate and the washing solution are united and concentrated to dryness. The obtained product is dried in a vacuum drying cabinet at 40° C. In this manner 8.1 g product were isolated. A determination of the composition of the product by the means of quantitative HPLC provided 64% N,N′,N″-Tris-(2-hydroyethyl)melamine, 16% N,N′-Di-(2-hydroxyethyl)melamine and 6% N-Mono-(2-hydroxyethyl)melamine and 14% N,N,N′,N″-Tetra-(2-hydroxyethyl)melamine.

### EXAMPLE 7

In a 1000 ml stirred autoclave 6 g N,N-dibutylamino-melamine, 250 g ethylenglycol and 10 g of a Ru/Al₂O₃ catalyst are intensively mixed so that the catalyst does not sink to the bottom. After closing the autoclave it is heated to 200° C. After 6 hours reaction time the reaction is aborted and the autoclave is cooled and depressurized. The cooled solution is separated from the catalyst via a filter and is concentrated in a high vacuum. The obtained product is dried in a vacuum drying cabinet at 40° C. In this manner 5.9 g product were isolated. A determination of the composition of the product by the means of quantitative HPLC provided 82% N,N-dibutylamino-N′,N″-di-(2-hydroxyethyl)-melamine, 15% N,N-dibutylamino-N′-mono-(2-hydroxyethyl)-melamine and 3% N,N,-dibutylamino-N′,N′,N″-tri-(2-hydroxyethyl)-melamine.

### EXAMPLE 8

In a 500 ml stirred autoclave 6 g N,N-dibutylamino-melamine, 20 g hydroxyacetic acid, 100 ml dimethoxyglycol and 8g of a Ni/Y-zeolith catalyst are intensively mixed so that the catalyst does not sink to the bottom. After locking the autoclave it is heated to 180° C. After 6 hours reaction time the reaction is aborted and the autoclave is cooled and depressurized. The solution is separated from the catalyst via a filter. The filtrate was taken up in 400m1 water/isopropanol (1:1) and the pH is adjusted to 6. The precipitated product is sucked off, washed and is dried in a vacuum drying cabinet at 40° C. In this manner 6.5 g product were isolated. A determination of the composition of the product by the means of quantitative HPLC provided 30% N,N-dibutylamino-melamine-N′-mono acetic acid, 68% N,N-dibutylamino-melamine-N′,N″-diacetic acid, 2% N, N-dibutylamino-melamine-N′,N′,N″-triacetic acid.

1-26. (canceled)

27. A method for producing a compound having at least one at least monosubstituted amino group, comprising
a starting substance having at least one amino group in form of a triazine derivative of the general formula (I) or a urea or urea derivative of the general formula (II): 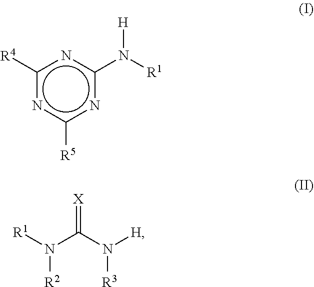
whereby
R⁴ and R⁵ mean independently from each other Q¹ or a moiety of the formula R⁶—N—R⁷ or R⁸—N—R⁹ connected with its central nitrogen atom to the triazine ring of the structure of formula (I), whereas
Q¹ means a linear or branched C₁-C₃₀-alkyl or a cyclic substituent in form of a C₅-C₂₀-cycloalkyl, a C₅-C₂₀-aryl, a C₁-C₂₀-alkylsubstituted C₅-C₂₀-aryl or an amide of a cyclic unsaturated carboxylic acid, whereat the C₁-C₃₀-alkyl or the cyclic substituent can be interrupted by one or multiple oxygen atoms, sulphur atoms, substituted nitrogen atoms and/or by one or multiple groups of the type —C(O)O—, —OC(O)—, —C(O)— and/or —OC(O)O—,
R¹, R², R³, R⁶, R⁷, R⁸ and R⁹ mean independently from each other H, linear or branched C₁-C₂₀-alkyl, C₅-C₂₀-cycloalkyl, C₅-C₂₀-aryl, C₁-C₂₀-alkylsubstituted C₅-C₂₀-aryl, which in each case can be interrupted by one or multiple oxygen atoms, sulphur atoms and/or substituted nitrogen atoms and/or by one or multiple groups of the type —C(O)O—, —OC(O)—, —C(O)— and/or —OC(O)— and/or can be functionalized by one or multiple hydroxyl groups and/or mercapto groups, and
X means Oor S
is reacted
with a reagent having a first functional group and at least one further functional group, in a reaction mixture, whereby the reagent has the general formula R¹⁰—OH so that at least one hydroxyl group is present as a first functional group in the reagent, whereas
R¹⁰ means a linear or branched C₁-C₂₀-alkyl, C₅-C₂₀-cycloalkyl or C₁-C₂₀-alkyl substituted C₅-C₂₀-aryl, which in each case can be interrupted by one or multiple oxygen atoms, sulphur atoms, substituted nitrogen atoms and/or by one or multiple groups of the type —C(O)O—, —OC(O)—, —C(O)— and/or —OC(O)O— and has at least one further functional group which is selected from the group consisting of hydroxyl groups, mercapto groups, carboxylic acid groups, carboxylic acid ester groups, carboxylic acid amide groups, keto groups, carbonate groups, epoxy groups, and carbamate groups, and whereby
the reaction is carried out in the presence of at least one further substance selected from the group consisting of ammonia, nitrogen, argon, and carbon monoxide.

28. The method according to claim 27, wherein the molar ratio of the further substance to the reagent is 0.1 to 2.

29. The method according to claim 27, wherein the reaction occurs at a total pressure of 1 to 200 bar.

30. The method according to claim 27, wherein the reaction mixture contains a catalyst.

31. The method according to claim 30, wherein the catalyst has a metal or a metal oxide or both.

32. The method according to claim 31, wherein the metal or the metal oxide or both comprises a metal from the 8^{th}, 9^{th} or 10^{th} group of the periodic system.

33. The method according to claim 31, wherein the catalyst has a carrier material.

34. The method according to claim 33, wherein the carrier material is a zeolithe, an alumo silicate, an alumo phosphate, a metal oxide, a silicate, a layered silicate, an aluminium oxide, silizium dioxide, or carbon.

35. The method according to claim 31, wherein the catalyst is separated after the completed reaction from the reaction mixture and is further processed.

36. The method according to claim 27, wherein the reaction occurs at a temperature of 100° C. to 300° C.

37. The method according to claim 27, wherein the reaction occurs during a reaction time of 1 minute to 100 hours.

38. The method according to claim 27, wherein at least one hydroxyl group of the reagent is present at the alkyl moiety and not at the aryl moiety, if R¹⁰ means a C₁-C₂₀ alkyl substituted C₅-C₂₀-aryl.

39. The method according to claim 27, wherein the produced compound is a triazine derivative of the general formula (III) or a urea derivative of the general formula (IV): 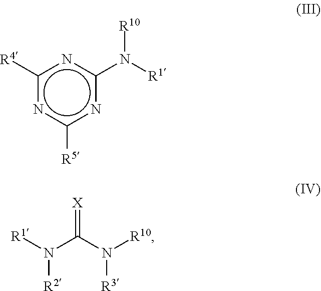
whereby
R^{4′} and R^{5′} mean independently from each other Q¹ or a moiety of the formula R^{6′}—N—R^{7′}— or R^{8′}—N—R^{9′} being connected with its central nitrogen atom to the triazine ring of the structure of the formula (III), whereas
Q¹ means a linear or branched C₁-C₃₀-alkyl or a cyclic substituent in form of a C₅-C₂₀-cycloalkyl, a C₅-C₂₀-aryl, a C₁-C₂₀-alkyl substituted C₅-C₂₀-aryl or an amide of a cyclic unsaturated carboxylic acid, whereas the C₁-C₃₀-alkyl or the cyclic substituent can be interrupted by one or multiple oxygen atoms, sulphur atoms, substituted nitrogen atoms and/or by one or multiple groups of the type —C(O)O—, —OC(O)—, —C(O)— and/or —OC(O)O—,
R^{1′}, R^{2′}, R^{3′}, R^{6′}, R^{8′} and R^{9′} mean independently from each other,
a covalent bond bound to the moiety R¹⁰ being bound to the same nitrogen atom of the triazine derivative or urea derivative so that a cyclic structure is formed from the nitrogen atom and the moiety R¹⁰,
H or
linear or branched C₁-C₂₀-alkyl, C₅-C₂₀-cycloalkyl, C₅-C₂₀-aryl, C₁-C₂₀-alkylsubstituted C₅-C₂₀-aryl, which in each case can be interrupted by one or multiple oxygen atoms, sulphur atoms and/or substituted nitrogen atoms and/or by one or multiple groups of the type —C(O)O—, —OC(O)—, —C(O)— and/or —OC(O)O— and/or can be functionalized by one or multiple hydroxyl groups and/or mercapto groups,
R¹⁰ means a linear or branched C₁-C₂₀-alkyl, C₅-C₂₀-cyclo alkyl or C₁-C₂₀-alkylsubstituted C₅-C₂₀-aryl, which in each case can be interrupted by one or multiple oxygen atoms, sulphur atoms, substituted nitrogen atoms and/or by one or multiple groups of the type —C(O)O—, —OC(O)—, —C(O)— and/or —OC(O)O—, and
a) has at least one further functional group, which is selected from the group consisting of hydroxyl groups, mercapto groups, carboxylic acid groups, carboxylic acid ester groups, carboxylic acid amide groups, keto groups, carbonate groups, halogens, epoxy groups, and carbamate groups, or
b) thereby forms a cyclic structure with two covalent bonds bonded to the same nitrogen atom of the compounds of the general formula (III) or (IV), whereat one of the two covalent bonds is provided by one of the moieties R^{1′}, R^{2′}, R^{3′}, R^{6′}, R^{7′}, R^{8′} or R^{9′} and whereat in the moiety R¹⁰ at least one further functional group can be present, which is selected from the group consisting of hydroxyl groups, mercapto groups, carboxylic acid groups, carboxylic acid ester groups, carboxylic acid amide groups, keto groups, carbonate groups, epoxy groups, and carbamate groups, and
X means O or S.

40. The method according to claim 39, wherein at least one moiety of the moieties R^{1′}, R^{2′}, R^{3′}, R^{6′}, R^{7′}, R^{8′} and R⁹′ have the meaning of the moiety R¹⁰.

41. The method according to claim 27, wherein the formed compound is selected from the group consisting of melamine-N-monoalkylcarboxylic acid, melamine-N,N′-dialkylcarboxylic acid, melamine-N,N′,N″-trialkylcarboxylic acid, melamine-N,N,N′,N″-tetra-alkylcarboxylic acid, melamine-N,N,N′,N′,N″-penta-alkylcarboxylic acid and melamine-N,N,N′,N′,N″,N″-hexa-alkylcarboxylic acid.

42. The method according to claim 27, wherein the formed compound is selected from the group consisting of urea-N-mono(alkylcarboxylic acid), urea-N,N′-di(alkylcarboxylic acid), urea-N,N,N′-tri(alkylcarboxylic acid), and urea-N,N,N′,N′-tetra(alkylcarboxylic acid).

43. The method according to claim 27, wherein the formed compound is selected from the group consisting of benzoguanamine-N-mono(alkylcarboxylic acid), benzoguanamine-N,N′-di(alkylcarboxylic acid), benzoguanamine-N,N,N′-tri(alkylcarboxylic acid), and benzoguanamine-N,N,N′,N′-tetra(alkylcarboxylic acid).

44. The method according to claim 27, wherein the formed compound is selected from the group consisting of acetoguanamine-N-mono(alkylcarboxylic acid), acetoguanamine-N,N′-di(alkylcarboxylic acid), acetoguanamine-N,N,N′-tri(alkylcarboxylic acid), and acetoguanamine-N,N,N′,N′-tetra (alkylcarboxylic acid).

45. The method according to claim 27, wherein the formed compound is selected from the group consisting of N,N′-Di-(hydroxyalkyl)-melamine, N,N′,N″-Tris-(hydroxyalkyl)-melamine, N,N,N′,N″-Tetra-(hydroxyalkyl)-melamine, N,N,N′,N′,N″-Penta-(hydroxyalkyl)-melamine, and N,N,N′,N′,N″,N″-Hexa-(hydroxyalkyl)-melamine.

46. The method according to claim 27, wherein the formed compound is selected from the group consisting of melamine-N-mono(alkylcarboxylic acid-alkylester), melamine-N,N′-di(alkylcarboxylic acid-alkylester), melamine-N,M,N″-tri(alkylcarboxylic acid-alkylester), melamine-N,N,N′,N″-tetra(alkylcarboxylic acid-alkylester), melamine-N,N,N′,N′,N″-penta(alkylcarboxylic acid-alkylester), and melamine-N,N,N′,N′,N″,N″-hexa(alkylcarboxylic acid-alkylester).

47. A method of providing oligomers, polymers, a formaldehyde resin, or a comonomer in aminoplast-formaldehyde-resins, comprising the step of producing a compound according to claim 27.

48. The method according to claim 47, wherein the formaldehyde resin is used as a laminate coating.

49. A method of providing a polyolefin containing mixture for improving the flame retardant properties or surface properties of an object, comprising the steps of producing a compound according to claim 27 and mixing the compound with a polyolefin.
